(19) United States
(12) Patent Application Publication
(10) Pub. No.: US 2019/0361024 A1
(43) Pub. Date: Nov. 28, 2019

(54) ENHANCED COLORECTAL CANCER SCREENING

(71) Applicant: CHECK-CAP LTD., Isfiya (IL)

(72) Inventor: Yoav KIMCHY, Haifa (IL)

(21) Appl. No.: 16/403,552

(22) Filed: May 5, 2019

Related U.S. Application Data
(60) Provisional application No. 62/676,992, filed on May 27, 2018.

(51) Int. Cl. G01N 33/574; A61B 5/06; A61B 10/00; A61B 1/04

(52) U.S. Cl. CPC G01N 33/57419; G01N 33/57484; A61B 6/481; A61B 10/0038; A61B 1/041; A61B 5/06

(57) ABSTRACT

A method of screening for colorectal cancer, including testing a person's feces, blood or urine for indications of colorectal cancer; if the results of the testing are negative scanning the walls of the colon with an X-ray imaging capsule to identify signs of abnormal growths; or scanning the walls of the colon with an X-ray imaging capsule to identify signs of abnormal growths; if the results of the scanning are negative testing a person's feces for indications of colorectal cancer. Wherein if the results of either the testing or the scanning are positive recommending examining the person with a colonoscopy examination, otherwise recommending retesting in a few years.

### RELATED APPLICATIONS

The present application claims priority from U.S. Provisional application No. 62/676,992 filed on May 27, 2018, the disclosure of which is incorporated herein by reference.

### TECHNICAL FIELD

The present disclosure relates generally to screening for colorectal cancer and more specifically selectively combining screening tests to enhance sensitivity with optimal specificity.

### BACKGROUND

There are currently a number of different methods for examining people to determine if they are healthy and do not have polyps or other abnormalities in the colon, which may indicate development of colorectal cancer. The most accurate method is performing a colonoscopy in which a practitioner inserts a flexible tube into the colon and with the aid of a light and camera examines the colon walls. However, every procedure has risks, for example the test may damage the colon or cause inflammation or infection. Likewise, the test has inconveniences since it requires preparations including restricting the subject's diet and taking laxatives beforehand. Additionally, the test is performed with sedation thus generally requiring somebody to accompany the subject to help them go home. Such a test typically interferes for a day or two with the subject's normal activities.

Therefore, simpler methods are sought to screen subjects by methods that present less of an interference with the subject's normal daily activities. One such method, which is simpler and non-invasive is known as a Fecal Immunochemical Test (FIT) that tests for occult blood in the stool. Blood in the stool may be a sign of colorectal cancer or other problems, such as polyps, ulcers or hemorrhoids. The results of the FIT test are not as conclusive as a colonoscopy, yet they may provide a first indication if a colonoscopy is required.

An additional method is known as Cologuard that tests a subject's stool for altered DNA from the cells lining the colon and/or blood in the stool.

Another method used is based on examining a blood sample, performing a “liquid biopsy” screening, looking for DNA in the blood or cells that have shed from a tumor and can be found in the blood stream. One such example of a blood screening test is EpiProColon from Epigenomics Inc (California).

Another method used is based on examining key metabolomics biomarkers in a urine sample. One such example of a urine screening test is PolypDx from Metabolomic Technologies Inc (Canada).

Another method, which requires only minimal preparations is based on the use of an X-ray based imaging capsule, such as described in U.S. Pat. No. 9,844,354 to Kimchy et al. The use of an imaging capsule typically requires drinking a contrast agent before swallowing the capsule but does not require cleaning out the colon before administration. The imaging capsule is capable of scanning the colon walls from the inside and reconstructing a 2D or 3D image of the colon walls.

Another method includes a swallowable capsule to perform a FIT test. U.S. provisional application 62/528,157 dated Jul. 3, 2017 the disclosure of which is incorporated herein by reference, discloses use of a swallowable capsule to perform a FIT test

The above screening methods are easier to administer than a colonoscopy however they are typically less accurate in detecting polyps or other abnormalities and may provide false positive or false negative results. It is thus desirable to find simple methods of colorectal cancer screening that improve overall sensitivity (true positive rate) of polyp detection without reducing specificity (true negative rate) and yet are applicable without extensive preparation.

### SUMMARY

An aspect of an embodiment of the disclosure relates to a system and method of screening a person for colorectal cancer. The system includes a kit for testing the person's feces, blood or urine for indications of the existence of polyps, tumors or other abnormal situations in the person's colon. The system further includes an imaging capsule to scan the walls of the person's colon internally and identify polyps or other abnormalities. The system further includes an external recorder or computer that communicates with the imaging capsule to record information from the capsule and display the images. The results of the feces, blood or urine test, and the results of the imaging capsule scanning can be recorded on the computer to provide recommendations for examining the person.

If the results of the feces, blood or urine test, or the results of the scanning are positive, then the system recommends that the person undergo a colonoscopy examination. In contrast if the feces, blood or urine test, or the imaging capsule scanning is negative then the other type (test or scan) needs to be performed. Only if both test (feces, blood or urine) and scan (imaging capsule) are negative then the person is assumed to be healthy and can wait a few years before retesting.

In some embodiments of the disclosure, the feces, blood or urine test will be performed first since it is generally simpler, quicker and cheaper. If the results are negative, then the image capsule is swallowed to scan the walls of the colon. Alternatively, the imaging capsule may be used first since it is more reliable than the feces, blood or urine test (since the imaging capsule physically scans the colon) and if, the results are negative, the feces, blood or urine test is performed to enhance accuracy of the determination of the system. In any case if the result is positive the person is recommended to perform a colonoscopy examination since there is a high probability that something needs to be dealt with internally, for example removing polyps.

There is thus provided according to an exemplary embodiment of the disclosure, a method of screening for colorectal cancer, comprising:

Testing a person's feces, blood or urine for indications of colorectal cancer; if the results of the testing are negative scanning the walls of the colon with an X-ray imaging capsule to identify signs of abnormal growths;

or

Scanning the walls of the colon with an X-ray imaging capsule to identify signs of abnormal growths; if the results of the scanning are negative testing a person's feces, blood or urine for indications of colorectal cancer;

Wherein if the results of either the testing or the scanning are positive recommending examining the person with a colonoscopy examination, otherwise recommending retesting in a few years.

In an exemplary embodiment of the disclosure, the testing includes a Fecal Immunochemical Test (FIT) that examines the feces for occult blood. Optionally, the testing includes a Cologuard Test that examines the feces for occult blood and checks for altered DNA from precancerous or cancerous cells. In an exemplary embodiment of the disclosure, the testing includes a Fecal Immunochemical Test (FIT) in a swallowable capsule. Optionally, the testing includes a blood test that identifies DNA resulting from the existence of colorectal tumors. In an exemplary embodiment of the disclosure, the testing includes a urine test that analyzes key metabolomics biomarkers in urine that indicate of the existence of colorectal tumors. Optionally, both the testing and the scanning are performed by a single swallowable capsule. In an exemplary embodiment of the disclosure, the capsule independently determines when and if to perform the testing and the scanning. In an exemplary embodiment of the disclosure, the capsule performs the testing and the scanning responsive to an instruction from an external controller. Optionally, the capsule performs the testing upon entering the colon to determine if to perform the scanning in the colon. In an exemplary embodiment of the disclosure, the capsule automatically performs the scanning and performs testing near the exit of the colon if the scanning was negative. Optionally, the capsule automatically performs the scanning and performs testing shortly after entering the colon if the initial scanning was negative.

There is further provided according to an exemplary embodiment of the disclosure, a system for screening for colorectal cancer, comprising:

A kit configured to perform testing of the feces, blood or urine of a person for indications of colorectal cancer;

A swallowable imaging capsule configured to perform scanning internal walls of the persons colon;

A computerized system that accepts the results from the imaging capsule and the kit, and configured to determine if the person should perform a colonoscopy examination or recommend retesting in a few years;

Wherein the determining is based on the results of either testing or scanning or both testing and scanning; if the results of either are positive then the colonoscopy examination is recommended; if the results of both are negative then retesting in a few years is recommended; if testing is performed first and the results are negative then scanning is recommended and if scanning is performed first then testing is recommended.

### BRIEF DESCRIPTION OF THE DRAWINGS

The present disclosure will be understood and better appreciated from the following detailed description taken in conjunction with the drawings. Identical structures, elements or parts, which appear in more than one figure, are generally labeled with the same or similar number in all the figures in which they appear, wherein:

FIG. 1 is a schematic illustration of a system for screening for colorectal cancer, according to an exemplary embodiment of the disclosure;

FIG. 2 is a flow diagram of a method of screening for colorectal cancer, according to an exemplary embodiment of the disclosure; and

FIG. 3 is a flow diagram of an alternative method of screening for colorectal cancer, according to an exemplary embodiment of the disclosure.

### DETAILED DESCRIPTION

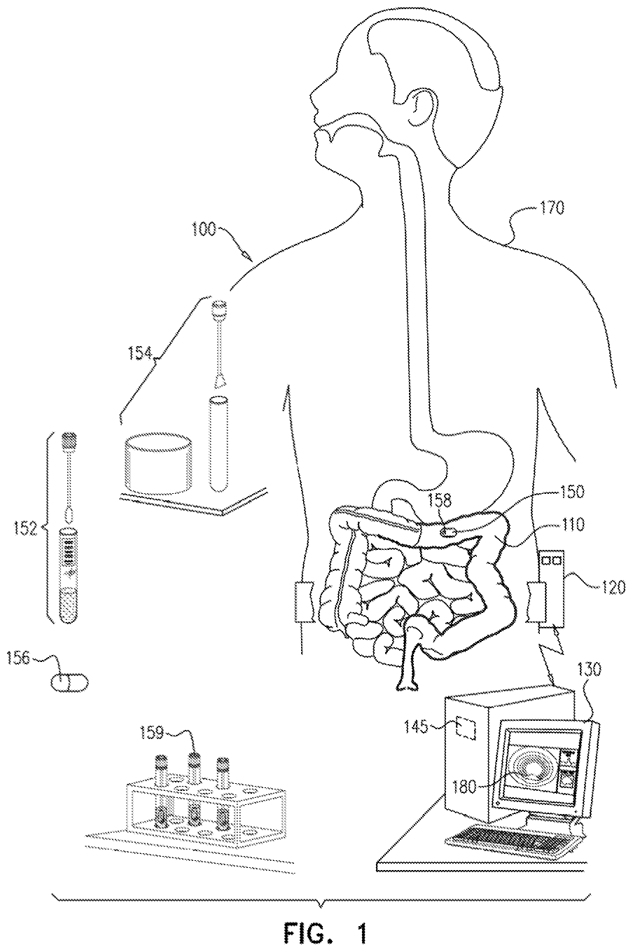

FIG. 1 is a schematic illustration of a system 100 for screening for colorectal cancer, according to an exemplary embodiment of the disclosure. System 100 includes an imaging capsule 150 that examines the internal walls of the gastrointestinal tract (including the colon 110) and a testing kit that examines the user's feces for the presence of occult blood and/or other indications of colorectal cancer. Alternatively, the testing kit may be in the form of a blood test to detect, the presence of altered DNA that indicates the presence of colorectal cancer tumors. Further alternatively, the testing kit may be in the form of a urine test that analyzes key metabolomics biomarkers in urine.

Optionally, the testing kit may be in the form of:

1. A Fecal Immunochemical Test (FIT) kit 152 that tests for occult blood by sampling the user's feces;

2. A Cologuard test kit 154 that checks for occult blood and also tests for altered DNA from precancerous or cancerous cells in the colon 110 by sampling the user's feces;

3. A FIT test kit 156 that is incorporated in a capsule 156 and is performed while traversing the colon 110. The FIT test 156 can sample at any position within the colon 110 based on the position of the capsule or external instructions. Optionally, the results of the FIT test can be transmitted to an external controller 120 immediately after determining the results while the capsule is still traveling through the colon 110; and

4. A Fit test kit 158 (similar to FIT test kit 156) combined with imaging capsule 150 in a single capsule;

5. A blood or urine test kit 159 to detect indicators of the existence of cancerous tumors in the colon based on analysis of the blood or urine.

In an exemplary embodiment of the disclosure, the testing kit is applied before using the imaging capsule or at the same time. In some embodiments of the disclosure, the testing kit may select and apply more than one test, e.g. feces and blood, or blood and urine. If all the selected tests from the kit provide a negative result then the result of the combined test is negative. If however one of the tests provide a positive result then the result of the combined test is positive.

Optionally, the imaging capsule 150 is ingested by a user 170 to traverse the user's gastrointestinal tract from the inside. In an exemplary embodiment of the disclosure, the imaging capsule 150 identifies its location with a tracking system. Optionally, the tracking system may identify its location independently or with the aid of an external controller 120 that communicates with the imaging capsule 150. In some embodiments of the disclosure capsule 150 may also use additional information to verify its location (e.g. based on time, current pressure, distance traveled).

In an exemplary embodiment of the disclosure, capsule 150 transmits information to external controller 120, which enables reconstruction of an image 180 of the internal walls of the colon 110. In some embodiments of the disclosure, external controller 120 may communicate with a computer 130 that executes an analysis application 145 to analyze the information received from the capsule 150 (e.g. identifying abnormalities) and provide information to the capsule 150 (e.g. to increase or decrease the sampling rate). In an exemplary embodiment of the disclosure, computer 130 receives measurements or location information from imaging capsule 150. The information may be analyzed to identify the current location of the capsule 150 and determine if the capsule 150 should perform any actions (e.g. perform a FIT test by sampling the content of the colon 110 at the current location of the imaging capsule 150). Optionally, computer 130 may provide or display images 180 of the inside of the colon 110 to a practitioner dealing with user 170. Likewise the computer can provide the results from measurements performed by the capsule 150 and recommendations for the user based on the results. In an exemplary embodiment of the disclosure, program 145 and recorded data can be stored on a non-volatile computer storage medium (e.g. CD, DVD or USB drive) to be transferred to other computers to analyze scan data and reconstruct images of a colon or a segment of a colon.

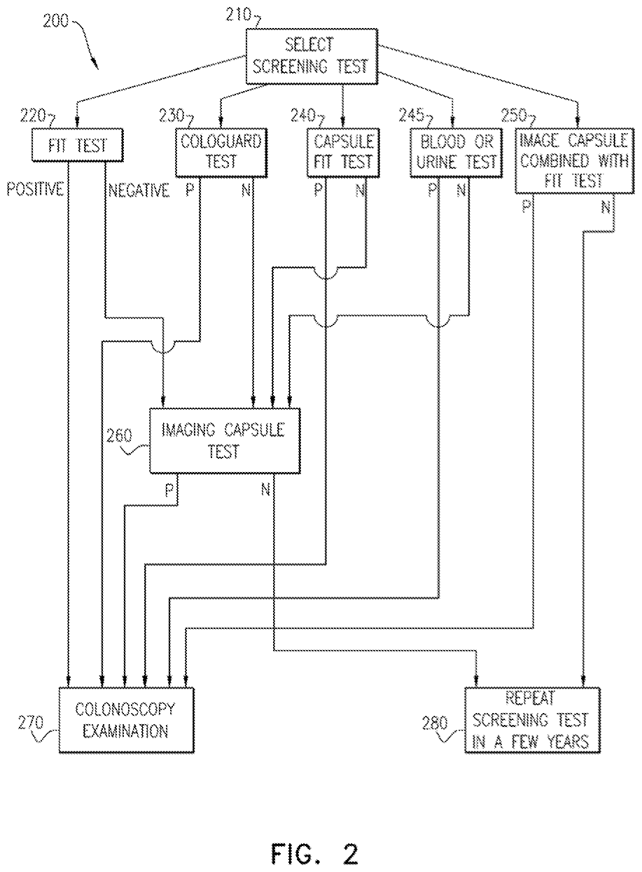

FIG. 2 is a flow diagram of a method 200 of screening for colorectal cancer, according to an exemplary embodiment of the disclosure. By empirical testing it has been shown that the accuracy of predicting the need or lack of need for a colonoscopy test is increased by combining the results from two simpler tests that do not requires extensive preparations, which could obstruct the normal daily lifestyle of the user. Each test examines different aspects and the combination of two simple tests improves overall sensitivity (the true positive rate) of polyp detection without reducing specificity (the true negative rate).

Initially a user selects (210) one or more basic screening tests, for example a FIT test (220), a Cologuard test (230), a capsule FIT test (240), a blood or urine test (245) or a combined test (250) using an image capsule 150 with a built in FIT test. Optionally, if any of the selected tests provides a positive result then the user should undergo a colonoscopy examination (270) since the user has polyps or abnormalities with a high, likelihood. If performing a colonoscopy examination then the imaging capsule scanning is unnecessary and there is no need to expose the user to radiation and cost of an imaging capsule 150.

If however the results of the Fit test (220), the Cologuard test (230), the capsule FIT test (240), or the blood or urine test (245) are negative then the user performs an imaging capsule test (260) that uses X-ray fluorescence and/or Compton backscattering to examine the inner walls of the colon 110 without the preparations required for a colonoscopy examination. The combined test (250) already includes imaging capsule 150 and screens the colon 110 while collecting and testing the user's feces with a built in FIT test.

In an exemplary embodiment of the disclosure, if the results of the tests are all negative then the user is assumed to be free from polyps or abnormalities in the colon and should repeat the testing process in a few years (280), for example in 3 to 5 years.

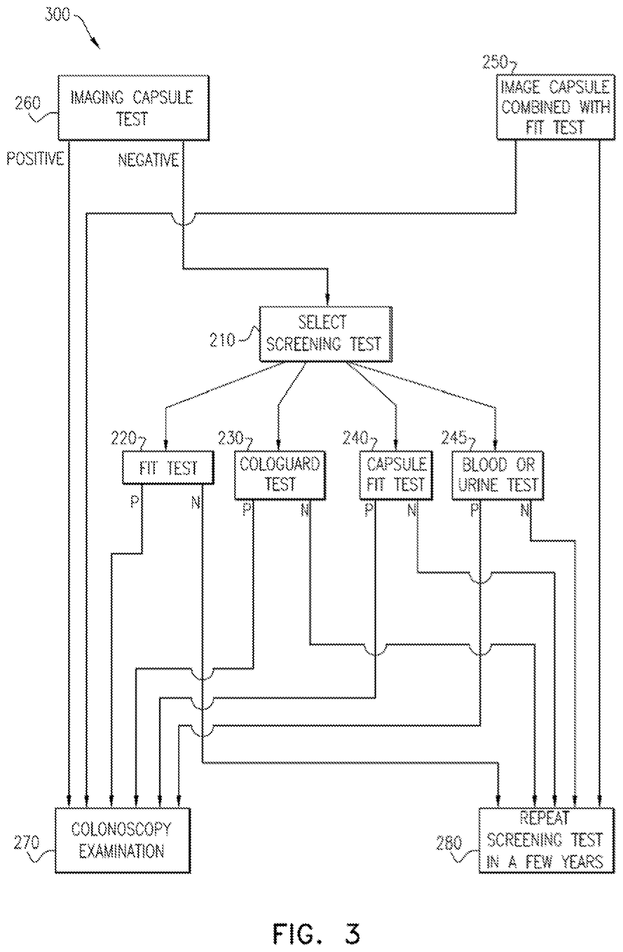

In some embodiments of the disclosure, scanning with an imaging capsule 150 is performed first and the feces, blood or urine are examined with one or more of the above testing kits (152, 154, 156, 159) if the results of the scanning are negative. FIG. 3 is a flow diagram of an alternative method 300 of screening for colorectal cancer, according to an exemplary embodiment of the disclosure.

In method 300 the user is first scanned with the image capsule test (260). If the results are positive the user is recommended to undergo a colonoscopy examination in which polyps may be removed. In contrast if the results are negative the user selects (210) one or more feces, blood or urine screening tests, for example a FIT test (220), a Cologuard test (230), a swallowable capsule that performs a FIT test inside the user's colon (240) or a blood or urine test (245). The feces, blood or urine screening test verifies that there is no need for a colonoscopy examination. If both tests were negative then it is highly probable that the user is healthy and the tests may be repeated in a few years. However if the feces, blood or urine test (220, 230, 240 or 245) or the scanning test (250) provide a positive result the user should undergo a colonoscopy examination to verify if there is a problem that triggered the positive result.

In an exemplary embodiment of the disclosure, when using the combined test (250) the FIT test 158 may be performed first and the X-ray scanning may be activated responsive to the results of the FIT test 158 to prevent X-ray scanning if a colonoscopy examination is required (e.g. the FIT test providing a positive result). Alternatively, the X-ray scanning may be performed first and the FIT test 158 performed near the end of the colon if the X-ray scanning is negative to enhance reliability of the result ruling out the need for a colonoscopy examination. If the results of the X-ray scanning with the imaging capsule 150 are positive the FIT test 158 is unnecessary since a colonoscopy examination is required.

Further alternatively, both the FIT testing and the X-ray scanning may be performed automatically regardless of the results of either and the final result, positive or negative is transmitted to the external controller 120. Optionally, the FIT test 158 may be performed upon entering the colon or near the exit of the colon. In some embodiments of the disclosure, the FIT test 158 may be performed more than once within the colon to enhance accuracy.

In an exemplary embodiment of the disclosure, external controller 120 may receive the results of the FIT test 158 performed by the imaging capsule 150 and instruct the capsule to start or stop scanning the colon. Alternatively, the external controller 120 may receive the results of the scanning and instruct the FIT test 158 in the capsule to be executed.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present disclosure is defined by the claims and not limited to what has been particularly shown and described hereinabove.

I/We claim:

1. A method of screening for colorectal cancer, comprising:
testing a person's feces, blood or urine for indications of colorectal cancer; if the results of the testing are negative scanning the walls of the colon with an X-ray imaging capsule to identify signs of abnormal growths;
or
scanning the walls of the colon with an X-ray imaging capsule to identify signs of abnormal growths; if the results of the scanning are negative testing a person's feces, blood or urine for indications of colorectal cancer;
wherein if the results of either the testing or the scanning are positive recommending examining the person with a colonoscopy examination, otherwise recommending retesting in a few years.

2. The method of claim 1, wherein the testing includes a Fecal Immunochemical Test (FIT) that examines the feces for occult blood.

3. The method of claim 1, wherein the testing includes a Cologuard Test that examines the feces for occult blood and checks for altered DNA from precancerous or cancerous cells.

4. The method of claim 1, wherein the testing includes a Fecal Immunochemical Test (FIT) in a swallowable capsule.

5. The method of claim 1, wherein the testing includes a blood test that identifies DNA resulting from the existence of colorectal tumors.

6. The method of claim 1, wherein the testing includes a urine test that analyzes key metabolomics biomarkers in urine that indicate of the existence of colorectal tumors.

7. The method of claim 1, wherein both the testing and the scanning are performed by a single swallowable capsule.

8. The method of claim 7, wherein the capsule independently determines when and if to perform the testing and the scanning.

9. The, method of claim 7, wherein the capsule performs the testing and the scanning responsive to an instruction from an external controller.

10. The method of claim 7, wherein the capsule performs the testing upon entering the colon to determine if to perform the scanning in the colon.

11. The method of claim 7, wherein the capsule automatically performs the scanning and performs testing near the exit of the colon if the scanning was negative.

12. The method of claim 7, wherein the capsule automatically performs the scanning and performs testing shortly after entering the colon if the initial scanning was negative.

13. A system for screening for colorectal cancer, comprising:
a kit configured to perform testing of the feces, blood or urine of a person for indications of colorectal cancer;
a swallowable imaging capsule configured to perform scanning internal walls of the persons colon;
a computerized system that accepts the results from the imaging capsule and the kit, and configured to determine if the person should perform a colonoscopy examination or recommend retesting in a few years;
wherein the determining is based on the results of either testing or scanning or both testing and scanning; if the results of either are positive then the colonoscopy examination is recommended; if the results of both are negative then retesting in a few years is recommended; if testing is performed first and the results are negative then scanning is recommended and if scanning is performed first then testing is recommended.

14. The system of claim 13, wherein the testing includes a Fecal Immunochemical Test (FIT) that examines the feces for occult blood.

15. The system of claim 13, wherein the testing includes a Cologuard Test that examines the feces for occult blood and checks for altered DNA from precancerous or cancerous cells.

16. The system of claim 13, wherein the testing includes a Fecal Immunochemical Test (FIT) in a swallowable capsule.

17. The system of claim 13, wherein the testing includes a blood test that identifies DNA resulting from the existence of colorectal tumors.

18. The system of claim 13, wherein the testing includes a urine test that analyzes key metabolomics biomarkers in urine that indicate of the existence of colorectal tumors.

19. The system of claim 13, wherein both the testing and the scanning are performed by a single swallowable capsule.

20. The system of claim 19, wherein the capsule independently determines when and if to perform the testing and the scanning.
